(19) United States
(12) Patent Application Publication
(10) Pub. No.: US 2026/0000693 A1
(43) Pub. Date: Jan. 1, 2026

(54) SEROTONERGIC PSYCHEDELIC AGENT FOR TREATING SELECTIVE MUTISM

(71) Applicant: LOBE SCIENCES LTD., Vancouver (CA)

(72) Inventors: Lyl TOMLINSON, Stewart, FL (US); Frederick SANCILIO, Stuart, FL (US); Maghsoud DARIANI, Fanwood, NJ (US)

(21) Appl. No.: 18/992,686

(22) PCT Filed: Jul. 12, 2023
(86) PCT No.: PCT/US2023/027475

§ 371 Date: Jan. 9, 2025

Related U.S. Application Data
(60) Provisional application No. 63/390,451, filed on Jul. 19, 2022.

(51) Int. Cl. A61K 31/675; A61K 31/198; A61K 31/4045

(52) U.S. Cl. CPC A61K 31/675; A61K 31/198; A61K 31/4045

(57) ABSTRACT

Methods and compositions for treating or alleviating symptoms of selective mutism in a human subject with a serotonergic psychedelic agent alone or in combination with N-acetylcysteine are provided.

This patent application claims the benefit or priority from U.S. Provisional Application Ser. No. 63/390,451, filed Jul. 19, 2022, teachings of which are incorporated by reference in their entirety.

### FIELD

This disclosure relates to methods and compositions for treating or alleviating symptoms of selective mutism (SM) in a human subject with a serotonergic psychedelic agent alone or in combination with N-acetylcysteine (NAC).

### BACKGROUND

Selective mutism (SM) is a rare childhood disorder, which presents as a persistent failure to speak in specific social situations even though speech production is normal in other contexts (Muris & Ollendick Psychology Research and Behavior Management 2021 14:159-167; Wong, P. Psychiatry 2010 7(3):23-31). For example, children with SM will often speak in their home in the presence of immediate family members but not in front of close friends or second-degree relatives, such as grandparents or cousins (American Psychiatric Association, 2013). Although SM can occur before the age of 5 (Viana et al. Clinical Psychology Review 2009 29(1):57-67), it usually does not come to clinical attention until the child starts school where there is an increase in social interaction and performance tasks. In such SM-priming contexts (e.g., school), afflicted children may rely on non-verbal cues like grunting, pointing, and writing in lieu of talking. They also display a variety of associated behavioral features: fear of social embarrassment, social isolation and withdrawal, clinging, compulsive traits, negativism, temper tantrums, and mild oppositional behavior (American Psychiatric Association, 2013). The disorder is relatively rare, showing a low prevalence in children with estimates varying between 0.03% and 1.9% depending upon the study (Viana et al. supra).

In clinical settings, children with SM are almost always given an additional diagnosis of another anxiety disorder with the most common being social anxiety disorder (American Psychiatric Association, 2013). However, SM can present with a variety of other comorbidities including obsessive-compulsive behaviors, elimination problems, depression, premorbid speech and language abnormalities and Asperger's disorder (Sharp et al. Journal of Anxiety Disorders 2007 21(4):568-579; Wong, P. supra). While the DSM V considers autism spectrum disorder (ASD) as an exclusion criterion of SM, it can often be difficult for clinicians to establish a clear boundary between these two conditions (Muris & Ollendick supra). Furthermore, increasing evidence has shown a clear relationship between SM and ASD. For example, one study found 62% of children with SM as the primary diagnosis could also have been diagnosed with ASD according to DSM standards (Steffenburg et al. Neuropsychiatric Disease and Treatment 2018 14:1163-1169). In another study, researchers found 80% of children with SM scored above the cut-off of a respected autism probability index (Klein et al. International Journal of Environmental Research and Public Health 2019 16(21):4070).

Previous reports indicate there is a high incidence of selective mutism in families with social phobias (Black & Uhde Journal of the American Academy of Child and Adolescent Psychiatry 1995 34(7):847-856) suggesting a common genetic predisposition for this disorder. In addition, rates of social anxiety disorder (SAD) and social anxiety traits are also markedly elevated in the families of children with ASD (Piven & Palmer American Journal of Psychiatry 1999 156(4):557-563; Smalley et al. American Journal of Medical Genetics-Neuropsychiatric Genetics 1995 60(1):19-26). Although a diagnosis of ASD would supersede a diagnosis of SM according to DSM-V, common features of impaired social interaction and communication problems raise questions about possible shared pathophysiology and a partially shared genetic basis for these symptoms. Recent studies have probed these links and found genetic variations in the CNTNAP2 (contactin-associated protein-like 2) gene which is expressed in CNS areas important for modulating higher order cognitive functions, including speech and language, reward and frontal executive function (Abrahams et al. PNAS 2007 104(45):17849-17845; Alarcón et al. American Journal of Human Genetics 2008 82(1):150-159). In line with this, disruptions of the CNTNAP2 gene have been reported in patients displaying a range of severe neurological disorders, and association studies have implicated variants within this locus in complex traits including language impairment and autism (Rodenas-Cuadrado et al. European Journal of Human Genetics 2014 22(2):171-178). Genetic variations in CNTNAP2 have even been associated with language-related deficits in healthy individuals (Whalley et al. American Journal of Medical Genetics 2011 156(8):941-948). More importantly, CNTNAP2 has specifically been linked with an increased risk for social anxiety-related traits and SM (Stein et al. Biological Psychiatry 2011 69(9):825-831).

CNTNAP2 knockout mouse models exhibit similar social behavior-related deficits, including repetitive behaviors and less time spent engaging in social play (Peñagarikano et al. Cell 2011 147(1):235-246). Researchers have used this mouse model to understand the neurobiological mechanisms and assess treatments for such disorders like ASD. CNTNAP2 is strongly expressed in oxytocin neurons and deleting the gene in mice reduces the total amount of brain oxytocin (Peñagarikano et al., Science Translational Medicine 2015 7:271). However, exogenous oxytocin is able to restore lost social behaviors in CNTNAP2 mice (Choe et al. Neuron 2022 110(5):765-808; Dölen et al. Nature 2013 501(7477):179-184; Peñagarikano et al. 2015 supra). Such restoration may occur from an increase in serotonin release activating downstream 5HT1B serotonin receptors (Dölen et al. supra).

Although there are nonmedication-based psychotherapeutic approaches for treating SM (i.e., psychodynamic therapy, behavioral therapy, and family therapy), few medication-based options exist with much of the focus being on selective serotonin reuptake inhibitors (SSRIs) (fluoxetine in particular) that have shown some success in improving SM symptoms (Manassis et al. European Child and Adolescent Psychiatry 2016 25(6):571-578; Wong, P. supra). Unfortunately, early-life exposure to SSRIs induces adult anxiety/depression-like phenotypes in rodents that are opposite to their mood-enhancing properties in adulthood (Rebello et al. Journal of Neuroscience 2014 34(37):12379-12393; Suri et al. Neuropsychopharmacology 2015 40(1):88-112). Similar issues have been reflected in pediatric patients and adolescents (Amitai et al. Current Treatment Option in Psychiatry 2015 291):28-37; Hammad et al. Archives of General Psychiatry 2006 63(3):332-339). SSRIs are thought to create these long-term deficits by persistently blocking a key serotonin transporter, Slc6a4/SERT (Soiza-Reilly et al. Molecular Psychiatry 2019 24(5):726-745).

There is a need for methods and compositions for treating and/or alleviating symptoms of SM.

### SUMMARY

An aspect of this disclosure relates to a method for alleviating one or more symptoms of selective mutism (SM) in a human. The method comprises administering to a human suffering from SM a serotonergic psychedelic agent.

In one nonlimiting embodiment, the method comprises administering a psilocibe-derived agent.

In one nonlimiting embodiment, the method further comprises administering to the human N-acetylcysteine (NAC).

In one nonlimiting embodiment, the human is a child suffering from SM.

Another aspect of this disclosure relates to compositions for use in alleviating one or more symptoms of SM comprising a serotonergic psychedelic agent.

In one nonlimiting embodiment, the composition comprises a psilocibe-derived agent.

In one nonlimiting embodiment, the composition further comprises NAC.

In one nonlimiting embodiment, the composition is formulated for administration to a child suffering from SM.

### DETAILED DESCRIPTION

Serotonergic psychedelic agents are a subset of hallucinogenics whose primary effect is to trigger non-ordinary states of consciousness (known as psychedelic experiences or “trips”) via serotonin 2A receptor agonism. This causes specific psychological, visual and auditory changes, and often a substantially altered state of consciousness. Psychedelics with the largest scientific and cultural influence include mescaline, lysergic acid diethylamide (LSD), psilocybin, and N, N-Dimethyltryptamine (DMT). Studies show that psychedelics are physiologically safe and do not lead to addiction (Nichols, D. E. Psychedelics Pharmacological reviews 2016 68 (2):264-355). Although further research is needed, existing results show that psychedelics may be useful for treating certain forms of psychopathology (Garcia-Romeu et al. Experimental and Clinical Psychopharmacology 2016 24 (4):229-268; Friedman, H. The Humanistic Psychologist 2006 34 (1):39-58; Tupper et al. CMAJ: Canadian Medical Association Journal 2015 187(14): 1054-1059).

For example, active ingredients in Psilocybe cubensis, psilocybin and/or psilocin create a sympathetic arousal state characterized by euphoria, visual and mental hallucinations, changes in perception, a distorted sense of time, spiritual experiences, giddiness, joy, open and closed eye visuals common at medium to high doses, along with synesthesia (e.g. hearing colors and seeing sounds). The mind-altering effects of psilocybin typically last from two to six hours. Adverse reactions include nausea, disorientation, lethargy and depression and panic attacks with about a third of users reporting feelings of anxiety or paranoia. Additional side effects include tachycardia, dilated pupils, restlessness or arousal, increased body temperature, headache, sweating and chills.

These effects are the result of psilocybin's rapid metabolism to psilocin, which then activates or partially activates several serotonin receptors including 5-HT2A, 5-HT2B and 5-HT2C in the brain. It is widely accepted that the hallucinogenic effects are generated primarily by agonist activity at the serotonin 5-HT2A receptor. Psilocin further binds with a slightly lower affinity to some 5-HT1 receptors, including 5-HT1A.

Psilocin has also been shown to bind to 5HT1B at a rate similar to the canonical 5HT2A receptor compared to most other monoamine receptors (Halberstadt & Geyer Neuropharmacology 2011 61(3):364-381).

The present disclosure provides methods and compositions for alleviating one or more symptoms of selective mutism in humans and in particular in children. The methods and compositions involve administration of a serotonergic psychedelic agent alone or in combination with N-acetylcysteine (NAC).

By “child” or “children” as used herein it is meant to include humans in early childhood through late adolescence.

As used herein, by “alleviating one or more symptoms of SM in a human”, it is meant to decrease one or more behaviors associated with SM including, but not limited to a persistent failure to speak in select social contexts, reliance on non-verbal cues like grunting, pointing, and writing in lieu of talking, fear of social embarrassment, social isolation and withdrawal, clinging, compulsive traits, negativism, temper tantrums, and mild oppositional behavior.

“Serotonergic psychedelic agent” as used herein, refers to a drug from the subset of hallucinogenic drugs whose primary effect is to trigger non-ordinary states of consciousness (known as psychedelic experiences or “trips”) via serotonin 5HT2A receptor agonism. Nonlimiting examples include mescaline, lysergic acid diethylamide (LSD), psilocybin or a psilocybin-derived agent, and N,N-Dimethyltryptamine (DMT).

In one nonlimiting embodiment, the psychedelic agent is psilocybin or a psilocybin-derived agent. Nonlimiting examples of psilocibe-derived agents which can be used in the methods and compositions of this disclosure include psilocybin and psilocin and salts, conjugates and esters thereof as well as 3,2-dimethylaminoethyl)-1H-indol-4-yl] dihydrogen phosphate, 4-hydroxytryptamine, 4-hydroxy-N,N-dimethyl-tryptamine, [3-(2-methylaminoethyl)-1H-indol-4-yl] dihydrogen phosphate, [3-(2-trimethylaminoethyl)-1H-indol-4-yl] dihydrogen phosphate and 4-hydroxy-N,N,N-trimethyltryptamine].

The inventors herein expect psilocybin or a psilocybin-derived agent to be able to avoid the pitfalls of SSRIs due to its different treatment mechanism. This expectation is bolstered by studies focused on serotonin receptor agonists (SRAs) which are used to treat chronic migraines in adults and children (Eiland & Hunt, Pediatric Drugs 2010 12(6):379-389). More specifically, many of the drugs within this class, including Frovitriptan Sumatriptan, Almotriptan (first FDA approved), and Lasmiditan are selective 5HT1B/1D receptor agonists, which have been deemed safe and effective with no serious adverse events reported (Charles, J. A. Journal of Headache and Pain 2006 7(2):95-97; Elkind et al. Journal of Clinical Pharmacology 2004 44(10):1158-1165; Tsai et al. Clinical Pharmacokinetics 2021 60(6):819-828). As some of these drugs have previously received governmental approval for use in adolescents, including Almotriptan (by the FDA in June 2009) and Sumatriptan (in Europe) (Eiland & Hunt, supra; Lewis, Expert Opinion on Pharmacotherapy 2010 11(14):2431-2436), the limited number of studies highlighting long-term, detrimental mental health effects in adolescents and children suggests that psilocin's mechanism of directly activating 5HT1B receptors, will also be less prone to causing those deleterious effects.

In one nonlimiting embodiment a soluble salt, conjugate or ester of psilocin is administered.

In one nonlimiting embodiment, a pantothenic salt of psilocin is administered.

In one nonlimiting embodiment, a mucate conjugate of psilocin is administered.

In one nonlimiting embodiment, a psilocin ester is administered. A nonlimiting example of a psilocin ester which can be administered is a psilocin-O-sulfate.

N-acetyl cysteine (NAC) is a potent antioxidant that also acts as a modulator of glutamatergic receptors, which may allow it to produce anti-depressive and anti-anxiety related effects. Adjunctive NAC treatment was also reported to address SSRI-inhibitor resistant anxiety in an adolescent case study.

In one nonlimiting embodiment, the psychedelic agent is administered alone to alleviate one or more symptoms of selective mutism.

In one nonlimited embodiment, the psychedelic agent is administered in combination with NAC to alleviate one or more symptoms of selective mutism.

In a preclinical rodent study, the combination of NAC and psilocybin was superior in reducing anxiety measures when compared to either drug alone. In accordance with this disclosure, the inventors herein believe similar results for SM can be obtained.

Preferred combination therapies in accordance with this disclosure are synergistic, meaning better than additive in their efficacy in alleviating one or more symptoms of SM.

As used herein, by “in combination” or “combination therapy” it is meant to include coadministration of the psychedelic agent and NAC, sequential administration of the psychedelic agent followed by NAC, or sequential administration of NAC followed by the psychedelic agent.

The psychedelic agent with or without NAC may be administered by any route providing for delivery of effective amounts to the brain. Examples of routes of administration include, but are in no way limited to, intravenous, intranasal, oral, topical, transdermal or via inhalation.

Doses and routes for administration for psychedelic agents will vary depending upon the psychedelic agent selected for administration. Selection may be based upon similar dosing regimens known in the art to be safe while exhibiting pharmacological activity. As a nonlimiting example, therapeutic ranges of 20 to 30 mg/70 kg of psilocybin have been disclosed. As will be understood by the skilled artisan upon reading this disclosure, similar dosing regimens to those already used for the psychedelic agent as well as alternative dosing regimens determined to be clinically relevant may be used.

In addition, psychedelic microdosing, a practice of using sub-threshold doses (microdoses) of serotonergic psychedelic drugs may be used.

Doses of NAC which have been administered safely for various conditions in humans range from 70 mg up to 6 grams per day. See webmd with the extension com/vitamins/ai/ingredientmono-1018/n-acetyl-cysteine-nac of the world wide web. As will be understood by the skilled artisan upon reading this disclosure, similar dosing regimens to those already used for NAC as well as alternative dosing regimens determined to be clinically relevant may be used.

In one nonlimiting embodiment, the psychedelic agent alone or the psychedelic agent and NAC are administered in a solid dosage formulation.

In one nonlimiting embodiment, the psychedelic agent alone or the psychedelic agent and NAC are administered as a tablet formulation.

In one nonlimiting embodiment, the psychedelic agent alone or the psychedelic agent and NAC are administered as a liquid formulation.

In one nonlimiting embodiment, an encapsulation technique is used to enclose various concentrations of the psychedelic agent with or without NAC in a relatively stable shell known as a capsule, allowing one or both agents to, for example, be taken orally. In one nonlimiting embodiment, the formulation of the present invention comprises a hard-shelled capsule containing dry, powdered ingredients, miniature pellets made by processes such as extrusion and spheronization or mini tablets. The hard-shelled capsules are typically made in two halves: a smaller-diameter body that is filled and then sealed using a larger-diameter cap. The capsule itself is typically made from aqueous solutions of gelling agents, such as animal protein (mainly gelatin) or plant polysaccharides or their derivatives (such as carrageenans and modified forms of starch and cellulose). Other ingredients can be added to the gelling agent solution including plasticizers such as glycerin or sorbitol to decrease the capsule's hardness, coloring agents, preservatives, disintegrants, lubricants and surface treatment.

In one nonlimiting embodiment, the psychedelic agent alone or the psychedelic agent and NAC are coadministered in a nasal spray formulation.

In one nonlimiting embodiment, psychedelic agent alone or the psychedelic agent and NAC are administered by nasal spray transducer programmed time release administration. A nonlimiting device for such administration is described in PCT/US2021/028068 filed Apr. 20, 2021, teachings of which are incorporated herein by reference in their entirety.

In one nonlimiting embodiment, the psychedelic agent alone or the psychedelic agent and NAC are administered in a nasal spray where therapeutically active amounts of psychedelic agent alone or the psychedelic agent and NAC are dissolved or suspended in solution or mixtures of excipients (e.g., preservatives, viscosity modifiers, emulsifiers, buffering agents) in nonpressurized dispensers that deliver a spray containing a metered dose of the therapeutically effective ingredient or ingredients.

1. A method for alleviating one or more symptoms of selective mutism (SM) in a human, said method comprising administering to a human suffering from SM a serotonergic psychedelic agent.

2. The method of claim 1 wherein the serotonergic psychedelic agent is a psilocibe-derived agent.

3. The method of claim 2 wherein the psilocibe-derived agent is psilocybin or psilocin or a salt, conjugate or ester thereof.

4. The method of claim 1 further comprising administering to the human N-acetylcysteine (NAC).

5. The method of claim 1 wherein the human is a child suffering from SM.

6. The method of claim 1 wherein one or more behaviors associated with SM is selected from a persistent failure to speak in select social contexts, reliance on non-verbal cues in lieu of talking, fear of social embarrassment, social isolation and withdrawal, clinging, compulsive traits, negativism, temper tantrums, and mild oppositional behavior is decreased.

7. A composition comprising a serotonergic psychedelic agent in an amount effective in alleviating one or more symptoms of SM.

8. The composition of claim 7 wherein the serotonergic psychedelic agent is a psilocibe-derived agent.

9. The composition of claim 8 wherein the psilocibe-derived agent is psilocybin or psilocin or a salt, conjugate or ester thereof.

10. The composition claim 7 further comprising human N-acetylcysteine (NAC).

11. The composition of claim 7 formulated for administration to a child suffering from SM.
